SEQUENCE LISTING (2 sequences)

SEQ ID NO: 1 - protein, 350 aa, Homo sapiens
MSNITDPQMWDFDDLNFTGMPPADEDYSPCMLETETLNKYVVIIAYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRHLVKFVCLGCWGLSMNLSLPFFLFRQAYHPNNSSPVCYEVLGNDTAKWRMVLRILPHTFGFIVPLFVMLFCYGFTLRTLFKAHMGQKHRAMRVIFAVVLIFLLCWLPYNLVLLADTLMRTQVIQESCERRNNIGRALDATEILGFLHSCLNPIIYAFIGQNFRHGFLKILAMHGLVSKEFLARHRVTSYTSSSVNVSSNL

SEQ ID NO: 2 - protein, 360 aa, Homo sapiens
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

(19) United States
(12) Patent Application Publication
(10) Pub. No.: US 2016/0017046 A1
(43) Pub. Date: Jan. 21, 2016

(54) COMPOSITIONS AND METHODS FOR TREATING OSTEOLYTIC BONE DISORDERS

(71) Applicant: MERRIMACK PHARMACEUTICALS, INC., Cambridge, MA (US)

(72) Inventors: Paul Kopesky, Lexington, MA (US); Brigit Schoeberl, Cambridge, MA (US)

(73) Assignee: Merrimack Pharmaceuticals, Inc., Cambridge, MA (US)

(21) Appl. No.: 14/770,752

(22) PCT Filed: Mar. 28, 2014
(86) PCT No.: PCT/US14/32165

§ 371 Date: Aug. 26, 2015

Related U.S. Application Data
(60) Provisional application No. 61/806,583, filed on Mar. 29, 2013.

(51) Int. Cl. C07K 16/28; A61K 45/06; A61K 39/395

(52) U.S. Cl. CPC C07K 16/2866; A61K 39/3955; A61K 45/06; C07K 2317/31; A61K 2039/507

(57) ABSTRACT

Provided are compositions comprising one of more molecules that specifically bind to CXC chemokine receptor 1 (CXCR1) and CXC chemokine receptor 2 (CXCR2) and methods for treating and improving the symptoms of pathologic bone loss in a subject by administering to the subject a therapeutically effective amount of such compositions.

### RELATED APPLICATIONS

This application is related to U.S. Provisional Patent Application No. 61/806,583, filed Mar. 29, 2013, the contents of which are incorporated herein by reference in its entirety.

### FIELD OF THE INVENTION

The present invention relates to compositions and methods useful for treating osteolytic bone disorders. More specifically, the invention relates to compositions comprising one of more molecules that specifically bind to CXC chemokine receptor 1 (CXCR1) and CXC chemokine receptor 2 (CXCR2) and methods for treating and improving the symptoms of pathologic bone loss in a subject by administering to the subject a therapeutically effective amount of such compositions.

### BACKGROUND

Pathologic bone loss (osteolysis) is one of the leading causes of morbidity worldwide. Several diseases and age-related conditions cause osteolysis, resulting in reduced bone mass (osteoporosis), bone and joint pain, and pathologic fractures. In the US alone, more than 18 million individuals will experience some bone loss due to osteoporosis, resulting in more than 1.5 million pathologic fractures. In addition, several human cancers metastasize to bone, resulting in osteolytic bone loss and its associated symptoms.

Accordingly, there exists an urgent need in the art to develop effective treatments for pathologic osteolysis.

### BRIEF DESCRIPTION OF THE FIGURES

FIG. 1 is a schematic illustration of the mechanisms of IL-8-mediated osteoclastogenesis and inhibition of IL-8 receptors with anti-CXCR1 and anti-CXCR2 antibodies.

FIG. 2 contains a first series of graphs that demonstrate the inhibition of osteoclastogenesis with antibodies blocking CXCR1 (FIG. 2A), CXCR2 (FIG. 2B), and IL-8 (FIG. 2C); and a second series of graphs characterizing the number of osteoclast precursors after CXCR1 and CXCR2 antibody treatment including the number of osteoclast precursors (FIG. 2D), the number of NFAT positive precursors (FIG. 2E) and the number of osteoclast nuclei (FIG. 2F).

FIG. 3 contains two graphs (A and B) that demonstrate the increased inhibition of osteoclastogenesis with combinations of anti-CXCR1 and anti-CXCR2 antibodies.

FIG. 4 is a graph that demonstrates the synergistic effect of anti-CXCR1 and anti-CXCR2 antibodies.

### SUMMARY OF THE INVENTION

The present invention relates to compositions and methods useful for treating osteolytic bone diseases and/or disorders. More specifically, the invention relates to compositions comprising one of more molecules that specifically bind to CXC chemokine receptor 1 (CXCR1) and CXC chemokine receptor 2 (CXCR2) and methods for treating and improving the symptoms of pathologic bone loss in a subject by administering to the subject a therapeutically effective amount of such compositions.

Accordingly, in one aspect, the invention is a composition comprising one or more molecules that specifically bind to CXC chemokine receptor 1 (CXCR1) and CXC chemokine receptor 2 (CXCR2). In one embodiment, at least one of the molecules of the invention is an antibody or an antigen binding fragment thereof. In another embodiment, the antibody is a bispecific antibody or a pan-specific antibody or an antigen binding fragment thereof.

In another embodiment, the composition comprises two antibodies or antigen binding fragments thereof. In another embodiment, the one or more molecules act synergistically to inhibit a common intracellular signaling pathway.

In one embodiment, the composition prevents interleukin-8 or another CXCR ligand from binding to CXCR1 and/or CXCR2. In another embodiment, the composition inhibits the activity of CXCR1 and/or CXCR2.

In one embodiment, the composition inhibits osteoclast differentiation and/or activity. In another embodiment, the composition promotes osteoblast activity. In another embodiment, the composition prevents bone resorption and/or promotes bone deposition. In yet another embodiment, the composition inhibits the growth of bone metastases in a subject.

In one embodiment, the composition further comprises an additional therapeutic agent. In another embodiment, the additional therapeutic agent is selected from the group consisting of a bisphosphonate, calcitonin, teriparatide, a parathyroid hormone analog, calcitonin, and a selective estrogen receptor modulator.

In another aspect, the invention provides a method for treating osteolysis in a subject in need thereof comprising administering to the subject a therapeutically effective amount of the composition of any one of the previous claims.

In one embodiment, following administration, the composition improves a bone parameter selected from the group consisting of bone volume density (BV/TV), total bone surface (BS), bone surface density (BS/BV), trabecular number (Tb.N), trabecular thickness (Tb.Th), trabecular spacing (Tb.Sp), and total volume (Dens TV).

In one embodiment, following administration, the composition reduces a serum biomarker of bone resorption selected from the group consisting of urinary hydroxyproline, urinary total pyridinoline (PYD), urinary free deoxypyridinoline (DPD), urinary collagen type-I cross-linked N-telopeptide (NTX), urinary or serum collagen type-I cross-linked C-telopeptide (CTX), bone sialoprotein (BSP), osteopontin (OPN), and tartrate-resistant acid phosphatase 5b (TRAP).

In one embodiment, following administration, the composition increases a serum biomarker of bone deposition selected from the group consisting of total alkaline phosphatase, bone-specific alkaline phosphatase, osteocalcin, and type-I procollagen (C-terminal/N-terminal).

In one embodiment, following administration, the composition inhibits bone resorption. In another embodiment, following administration, the composition promotes bone deposition. In another embodiment, following administration, the composition inhibits the growth of bone metastases in a subject. In yet another embodiment, following administration, the composition improves a symptom of a subject with bone metastases.

In one embodiment, the administering of the composition is by a parenteral or an oral route. In another embodiment, the parenteral route is a subcutaneous, intradermal, intramuscular, intraperitoneal, intravenous, intranasal, intrathecal, inhalation, or intrarticular route.

### DETAILED DESCRIPTION OF THE INVENTION

### A. Definitions

Unless defined otherwise, all technical and scientific terms used herein have the same meaning as is commonly understood by one of ordinary skill in the art.

It is noted here that as used in this specification and the appended claims, the singular forms “a”, “an”, and “the” also include plural reference, unless the context clearly dictates otherwise.

The term “about” or “approximately” means within 10%, and more preferably within 5% (or 1% or less), of a given value or range.

The terms “administer” or “administration” refer to the act of injecting or otherwise physically delivering a substance as it exists outside the body (e.g., an antibody) into a patient, such as by mucosal, intradermal, intravenous, subcutaneous, intramuscular delivery, and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

The term “CXCR1” refers to chemokine (C-X-C) receptor 1, the term “CXCR2” refers to chemokine (C-X-C) receptor 2, and the term “CXCR1/2” refers to both CXCR1 and CXCR2. The term “IL-8” refers to interleukin-8.

An “antagonist” or “inhibitor” of CXCR1/2 refers to one or more molecules that are capable of inhibiting or otherwise decreasing one or more of the biological activities of CXCR1/2, such as in a cell expressing CXCR1/2 or in a cell expressing a CXCR1/2 ligand (e.g., IL-8). In certain exemplary embodiments, one or more antibodies of the invention are antagonist antibodies that inhibit or otherwise decrease the activity of CXCR1/2 in a cell having a cell surface-expressed CXCR1/2 receptor (e.g., CXCR1 or CXCR2) when said antibody is contacted with said cell. In some embodiments, an antagonist of CXCR1/2 (e.g., an antibody of the invention) may, for example, act by inhibiting or otherwise decreasing the activation and/or cell signaling pathways of the cell expressing a CXCR1 and/or CXCR2 receptor, thereby inhibiting a CXCR1/2-mediated biological activity of the cell relative to the CXCR1/2-mediated biological activity in the absence of antagonist. In certain embodiments of the invention, the one or more anti-CXCR1/2 antibodies are antagonistic anti-CXCR1/2 antibodies, preferably fully human, monoclonal, antagonistic anti-CXCR1/2 antibodies.

The terms “antibody”, “immunoglobulin”, or “Ig” may be used interchangeably herein. The term antibody includes, but is not limited to, synthetic antibodies, monoclonal antibodies, recombinantly produced antibodies, multispecific antibodies (including bispecific antibodies), human antibodies, humanized antibodies, chimeric antibodies, intrabodies, single-chain Fvs (scFv) (e.g., including monospecific, bispecific, etc.), camelized antibodies, Fab fragments, F(ab′) fragments, disulfide-linked Fvs (sdFv), anti-idiotypic (anti-Id) antibodies, and epitope-binding fragments of any of the above. In particular, antibodies include immunoglobulin molecules and immunologically active portions of immunoglobulin molecules, i.e., antigen binding domains or molecules that contain an antigen-binding site that specifically binds to a CXCR1/2 antigen (e.g., one or more complementarity determining regions (CDRs) of an anti-CXCR1/2 antibody). The anti-CXCR1/2 antibodies can be of any type (e.g., IgG, IgE, IgM, IgD, IgA, and IgY), any class (e.g., IgG1, IgG2, IgG3, IgG4, IgA1, and IgA2), or any subclass (e.g., IgG2a and IgG2b) of an immunoglobulin molecule. In certain embodiments, the anti-CXCR1/2 antibodies are humanized, such as humanized monoclonal anti-CXCR1/2 antibodies. In other embodiments, the anti-CXCR1/2 antibodies are fully human, such as fully human monoclonal anti-CXCR1/2 antibodies.

The terms “composition” and “formulation” are intended to encompass a product containing specified ingredients (e.g., an anti-CXCR1/2 antibody or antibodies) in, optionally, specified amounts, as well as any product which results, directly or indirectly, from the combination of specified ingredients in, optionally, specified amounts.

The terms “constant region” or “constant domain” refer to a carboxy terminal portion of the light and heavy chain that is not directly involved in binding of the antibody to antigen, but exhibits various effector functions, such as interaction with the Fc receptor. The terms refer to the portion of an immunoglobulin molecule having a more conserved amino acid sequence relative to the other portion of the immunoglobulin, the variable domain, which contains the antigen binding site. The constant domain contains the CH1, CH2, and CH3 domains of the heavy chain, and the CHL domain of the light chain.

The term “epitope” refers to a localized region on the surface of an antigen, such as a CXCR1/2 polypeptide or CXCR1/2 polypeptide fragment, that is capable of being bound to one or more antigen binding regions of an antibody, and that has antigenic or immunogenic activity in an animal, preferably a mammal, and most preferably in a human, that is capable of eliciting an immune response. An epitope having immunogenic activity is a portion of a polypeptide that elicits an antibody response in an animal. An epitope having antigenic activity is a portion of a polypeptide to which an antibody specifically binds, as determined by any method well known in the art, for example, such as an immunoassay. Antigenic epitopes need not necessarily be immunogenic. Epitopes usually consist of chemically active surface groupings of molecules, such as amino acids or sugar side chains, and have specific three-dimensional structural characteristics, as well as specific charge characteristics. A region of a polypeptide contributing to an epitope may be contiguous amino acids of the polypeptide or the epitope may come together from two or more non-contiguous regions of the polypeptide. The epitope may or may not be a three-dimensional surface feature of the antigen. In certain embodiments, a CXCR1/2 epitope is a three-dimensional surface feature of a CXCR1/2 polypeptide. In other embodiments, a CXCR1/2 epitope is a linear feature of a CXCR1/2 polypeptide. Anti-CXCR1/2 antibodies may specifically bind to a three dimensional or linear epitope of CXCR1/2.

The term “excipients” refers to inert substances that are commonly used as a diluent, vehicle, preservative, binder, stabilizing agent, etc. for drugs and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, Remington's Pharmaceutical Sciences (1990) Mack Publishing Co., Easton, Pa.

In the context of a peptide or polypeptide, the term “fragment” refers to a peptide or polypeptide that comprises less than the full length amino acid sequence. Such a fragment may arise, for example, from a truncation at the amino terminus, a truncation at the carboxy terminus, and/or an internal deletion of a residue(s) from the amino acid sequence. Fragments may, for example, result from alternative RNA splicing or from in vivo protease activity. In certain embodiments, CXCR1/2 fragments include polypeptides comprising an amino acid sequence of at least 50, at 100 amino acid residues, at least 125 contiguous amino acid residues, at least 150 contiguous amino acid residues, at least 175 contiguous amino acid residues, at least 200 contiguous amino acid residues, at least 250 contiguous amino acid residues, at least 300 contiguous amino acid residues, or at least 350 contiguous amino acid residues of the amino acid sequence of a CXCR1/2 polypeptide. In a specific embodiment, a fragment of a CXCR1/2 polypeptide or an antibody that specifically binds to a CXCR1/2 antigen retains at least 1, at least 2, or at least 3 functions of the full-length polypeptide or antibody.

The terms “fully human antibody” or “human antibody” are used interchangeably herein and refer to an antibody that comprises a human variable region and, most preferably a human constant region. In specific embodiments, the terms refer to an antibody that comprises a variable region and constant region of human origin. “Fully human” anti-CXCR1/2 antibodies, in certain embodiments, can also encompass antibodies that bind CXCR1/2 polypeptides and are encoded by nucleic acid sequences that are naturally occurring somatic variants of a human germline immunoglobulin nucleic acid sequence. In a specific embodiment, the anti-CXCR1/2 antibodies are fully human antibodies. The term “fully human antibody” includes antibodies having variable and constant regions corresponding to human germline immunoglobulin sequences as described by Kabat et al. (See Kabat et al. (1991) Sequences of Proteins of Immunological Interest, Fifth Edition, U.S. Department of Health and Human Services, NIH Publication No. 91-3242). Methods of producing fully human antibodies are known in the art.

The phrase “recombinant human antibody” includes human antibodies that are prepared, expressed, created, or isolated by recombinant means, such as antibodies expressed using a recombinant expression vector transfected into a host cell, antibodies isolated from a recombinant, combinatorial human antibody library, antibodies isolated from an animal (e.g., a mouse or cow) that is transgenic and/or transchromosomal for human immunoglobulin genes (see, e.g., Taylor, L. D. et al. (1992) Nucl. Acids Res. 20:6287-6295) or antibodies prepared, expressed, created, or isolated by any other means that involves splicing of human immunoglobulin gene sequences to other DNA sequences. Such recombinant human antibodies can have variable and constant regions derived from human germline immunoglobulin sequences (See Kabat, E. A. et al. (1991) Sequences of Proteins of Immunological Interest, Fifth Edition, U.S. Department of Health and Human Services, NIH Publication No. 91-3242). In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “heavy chain” when used in reference to an antibody refers to five distinct types, called alpha (α), delta (Δ), epsilon (ε), gamma (γ) and mu (μ), based on the amino acid sequence of the heavy chain constant domain. These distinct types of heavy chains are well known in the art and give rise to five classes of antibodies, IgA, IgD, IgE, IgG, and IgM, respectively, including four subclasses of IgG, namely IgG1, IgG1, IgG3, and IgG4. Preferably the heavy chain is a human heavy chain.

An “isolated” or “purified” antibody is substantially free of cellular material or other contaminating proteins from the cell or tissue source from which the antibody is derived, or substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, or 5% (by dry weight) of heterologous protein (also referred to herein as a “contaminating protein”). When the antibody is recombinantly produced, it is also preferably substantially free of culture medium, i.e., culture medium represents less than about 20%, 10%, or 5% of the volume of the protein preparation. When the antibody is produced by chemical synthesis, it is preferably substantially free of chemical precursors or other chemicals, i.e., it is separated from chemical precursors or other chemicals that are involved in the synthesis of the protein. Accordingly, such preparations of the antibody have less than about 30%, 20%, 10%, or 5% (by dry weight) of chemical precursors or compounds other than the antibody of interest. In a preferred embodiment, anti-CXCR1/2 antibodies are isolated or purified.

The terms “Kabat numbering,” and like terms are recognized in the art and refer to a system of numbering amino acid residues that are more variable (i.e. hypervariable) than other amino acid residues in the heavy and light chain variable regions of an antibody, or an antigen binding portion thereof (Kabat et al. (1971) Ann NY Acad. Sci. 190:382-391 and, Kabat et al. (1991) Sequences of Proteins of Immunological Interest, Fifth Edition, U.S. Department of Health and Human Services, NIH Publication No. 91-3242). For the heavy chain variable region, the hypervariable region typically ranges from amino acid positions 31 to 35 for CDR1, amino acid positions 50 to 65 for CDR2, and amino acid positions 95 to 102 for CDR3. For the light chain variable region, the hypervariable region typically ranges from amino acid positions 24 to 34 for CDR1, amino acid positions 50 to 56 for CDR2, and amino acid positions 89 to 97 for CDR3.

The term “light chain” when used in reference to an antibody refers to two distinct types, called kappa (κ) of lambda (λ), based on the amino acid sequence of the constant domains. Light chain amino acid sequences are well known in the art. In preferred embodiments, the light chain is a human light chain.

The terms “manage”, “managing”, and “management” refer to the beneficial effects that a subject derives from a therapy (e.g., a prophylactic or therapeutic agent), which does not result in a cure of the disease or disorder. In certain embodiments, a subject is administered one or more therapies (e.g., prophylactic or therapeutic agents) to “manage” a CXCR1/2-mediated disease (e.g., pathologic osteolysis), or one or more symptoms thereof, so as to prevent the progression or worsening of the disease.

The term “monoclonal antibody” refers to an antibody obtained from a population of homogenous or substantially homogeneous antibodies, and each monoclonal antibody will typically recognize a single epitope on the antigen. In preferred embodiments, a “monoclonal antibody” is an antibody produced by a single hybridoma or other cell. The term “monoclonal” is not limited to any particular method for making the antibody. For example, monoclonal antibodies may be made by the hybridoma method as described in Kohler et al.; Nature, 256:495 (1975) or may be isolated from phage libraries. Other methods for the preparation of clonal cell lines and of monoclonal antibodies expressed thereby are well known in the art (see, for example, Chapter 11 in: Short Protocols in Molecular Biology, (2002) 5th Ed.; Ausubel et al., eds., John Wiley and Sons, New York).

The term “pharmaceutically acceptable” means being approved by a regulatory agency of the Federal or a State government or listed in the U.S. Pharmacopeia, European Pharmacopeia, or other generally recognized Pharmacopeia for use in animals, and more particularly in humans.

The term “pharmaceutically acceptable excipient” means any inert substance that is combined with an active molecule, such as a monoclonal antibody, for preparing an agreeable or convenient dosage form. The “pharmaceutically acceptable excipient” is an excipient that is non-toxic to recipients at the dosages and concentrations employed, and is compatible with other ingredients of the formulation comprising the monoclonal antibody.

The terms “prevent”, “preventing”, and “prevention” refer to the total or partial inhibition of the development, recurrence, onset, or spread of a CXCR1/2-mediated disease and/or symptom related thereto, resulting from the administration of a therapy or combination of therapies provided herein (e.g., a combination of prophylactic or therapeutic agents).

The term “CXCR1/2 antigen” refers to that portion of a CXCR1/2 polypeptide to which one or more binding agents, such as an antibody or a combination of antibodies specifically binds. A CXCR1/2 antigen also refers to an analog or derivative of a CXCR1/2 polypeptide or fragment thereof to which an antibody specifically binds. In some embodiments, a CXCR1/2 antigen is a monomeric CXCR1/2 antigen or a dimeric CXCR1/2 antigen. A region of a CXCR1/2 polypeptide contributing to an epitope may be contiguous amino acids of the polypeptide, or the epitope may come together from two or more non-contiguous regions of the polypeptide. The epitope may or may not be a three-dimensional surface feature of the antigen. A localized region on the surface of a CXCR1/2 antigen that is capable of eliciting an immune response is a CXCR1/2 epitope. As used herein, an “analog” of the CXCR1/2 antigen refers to a polypeptide that possesses a similar or identical function as a CXCR1/2 polypeptide, a fragment of a CXCR1/2 polypeptide, or a CXCR1/2 epitope described herein. For example, the analog may comprise a sequence that is at least 80%, at least 85%, at least 90%, at least 95%, or at least 99% identical to the amino acid sequence of a CXCR1/2 polypeptide (e.g., SEQ ID NO: 1 or SEQ ID NO:2), a fragment of a CXCR1/2 polypeptide, a CXCR1/2 epitope, or an anti-CXCR1/2 antibody described herein. Additionally or alternatively, the polypeptide is encoded by a nucleotide sequence that hybridizes under stringent conditions to a nucleotide sequence encoding a CXCR1/2 polypeptide, a fragment of a CXCR1/2 polypeptide, or a CXCR1/2 epitope described herein.

The terms “human CXCR1/2,” “hCXCR1/2,” or “CXCR1/2 polypeptide” and similar terms refer to the polypeptides (“polypeptides,” “peptides,” and “proteins” are used interchangeably herein) comprising the amino acid sequences of SEQ ID NO: 1 and SEQ ID NO:2, and related polypeptides, including SNP variants thereof. Related polypeptides include allelic variants (e.g., SNP variants); splice variants; fragments; derivatives; substitution, deletion, and insertion variants; fusion polypeptides; and interspecies homologs, preferably, which retain CXCR1/2 activity and/or are sufficient to generate an anti-CXCR1/2 immune response. Also encompassed are soluble forms of CXCR1/2 that are sufficient to generate an anti-CXCR1/2 immunological response. As those skilled in the art will appreciate, an anti-CXCR1/2 antibody can bind to a CXCR1/2 polypeptide, polypeptide fragment, antigen, and/or epitope, as an epitope is part of the larger antigen, which is part of the larger polypeptide fragment, which, in turn, is part of the larger polypeptide. hCXCR1/2 can exist in a dimeric or monomeric form.

The terms “CXCR1/2-mediated disease” and “CXCR1/2-mediated disorder” are used interchangeably and refer to any disease or disorder that is completely or partially caused by or is the result of CXCR1/2, e.g., hCXCR1/2. In certain embodiments, CXCR1/2 is aberrantly expressed. In other embodiments, IL-8 is aberrantly expressed. In some embodiments, CXCR1/2 or IL-8 may be aberrantly upregulated in a particular cell type. In other embodiments, normal, aberrant, or excessive cell signaling is caused by binding of IL-8 to CXCR1/2 receptors. In certain embodiments, IL-8 receptors (e.g., CXCR1/2 receptors), are expressed on the surface of a cell, such as an osteoblast, osteoclast, or a precursor of either cell type. In certain embodiments, the CXCR1/2-mediated disease is a degenerative bone disease, such as pathologic osteolysis.

The terms “specifically binds” or “specifically binding” mean specifically binding to an antigen or a fragment thereof (e.g., CXCR1/2) and not specifically binding to other antigens. An antibody that specifically binds to an antigen may bind to other peptides or polypeptides with lower affinity, as determined by, e.g., radioimmunoassays (RIA), enzyme-linked immunosorbent assays (ELISA), BIACORE, or other assays known in the art. In certain embodiments, an anti-CXCR1/2 antibody of the invention may specifically bind to CXCR1/2 (e.g., hCXCR1/2) with more than two-fold greater affinity than a different, non-CXCR1/2 antigen. Antibodies or variants or fragments thereof that specifically bind to an antigen may be cross-reactive with related antigens. For example, in certain embodiments an anti-CXCR1/2 antibody may cross-react with hCXCR1/2 and another CXCR1/2 antigen (e.g., a rodent or non-human primate CXCR1/2 antibody). Preferably, antibodies or variants or fragments thereof that specifically bind to an antigen do not cross-react with other non-CXCR1/2 antigens. An antibody or a variant or a fragment thereof that specifically binds to a CXCR1/2 antigen can be identified, for example, by immunoassays, BIAcore, or other techniques known to those of skill in the art. Typically, a specific or selective reaction will be at least twice background signal or noise, and more typically more than 10 times background. In some embodiments, the binding protein or antibody will bind to its antigen, e.g. CXCR1/2, with a dissociation constant of between 1×10⁻⁶ M and 1×10⁻⁷. In other embodiments, the dissociation constant is between 1×10⁻⁶M and 1×10⁻⁸. See, e.g., Paul, ed., 1989, Fundamental Immunology Second Edition, Raven Press, New York at pages 332-336 for a discussion regarding antibody specificity.

The terms “subject” and “patient” are used interchangeably. As used herein, a subject is preferably a mammal, such as a non-primate (e.g., cows, pigs, horses, cats, dogs, rats, etc.) or a primate (e.g., monkey and human), most preferably a human. In one embodiment, the subject is a mammal, preferably a human, having a CXCR1/2-mediated disease. In another embodiment, the subject is a mammal, preferably a human, at risk of developing a CXCR1/2-mediated disease.

The term “therapeutic agent” refers to any agent that can be used in the treatment, management, or amelioration of a CXCR1/2-mediated disease and/or a symptom related thereto. In certain embodiments, the term “therapeutic agent” refers to a CXCR1/2 antibody. In certain other embodiments, the term “therapeutic agent” refers to an agent other than a CXCR1/2 antibody. Preferably, a therapeutic agent is an agent that is known to be useful for, or has been, or is currently being used for the treatment, management, or amelioration of a CXCR1/2-mediated disease, or one or more symptoms related thereto.

The term “therapy” refers to any protocol, method, and/or agent that can be used in the prevention, management, treatment, and/or amelioration of a CXCR1/2-mediated disease (e.g., pathologic osteolysis). In certain embodiments, the terms “therapies” and “therapy” refer to a biological therapy, supportive therapy, and/or other therapies useful in the prevention, management, treatment, and/or amelioration of a CXCR1/2-mediated disease known to one of skill in the art, such as medical personnel.

The terms “treat”, “treatment”, and “treating” refer to the reduction or amelioration of the progression, severity, and/or duration of a CXCR1/2-mediated disease (e.g., pathologic osteolysis) resulting from the administration of one or more therapies (including, but not limited to, the administration of one or more prophylactic or therapeutic agents). In specific embodiments, such terms refer to the reduction or inhibition of the binding of IL-8 to a CXCR1/2 receptor, the reduction or inhibition of the production or secretion of IL-8 from a cell expressing a CXCR1/2 receptor of a subject, the reduction or inhibition of the production or secretion of IL-8 from a cell not expressing a CXCR1/2 receptor of a subject, inhibition of the activity of a CXCR1/2 receptor expressed by a cell, and/or the inhibition or reduction of one or more symptoms associated with a CXCR1/2-mediated disease, such as pathologic osteolysis.

The terms “variable region” or “variable domain” refer to a portion of the light and heavy chains, typically about the amino-terminal 120 to 130 amino acids in the heavy chain and about 100 to 110 amino acids in the light chain, which differ extensively in sequence among antibodies and are used in the binding and specificity of each particular antibody for its particular antigen. The variability in sequence is concentrated in those regions called complementarity determining regions (CDRs), while the more highly conserved regions in the variable domain are called framework regions (FR). The CDRs of the light and heavy chains are primarily responsible for the interaction of the antibody with antigen. Numbering of amino acid positions is according to the EU Index, as in Kabat et al. (1991) Sequences of proteins of immunological interest. (U.S. Department of Health and Human Services, Washington D.C.) 5^{th} ed. (“Kabat et al.”). In preferred embodiments, the variable region is a human variable region.

### B. Bone Biology

The vertebrate skeleton is comprised of bone, which is a living, calcified tissue that provides structure, support, protection, and a source of minerals for regulating ion transport. Bone is a specialized connective tissue that is comprised of both cellular and acellular components. The acellular extracellular matrix (ECM) contains both collagenous and non-collagenous proteins, both of which participate in the calcification process.

The terms “cortical bone” or “compact bone” refer to the outer layer of bone, which is dense, rigid, and tough. The terms “trabecular bone” or “cancellous bone” refer to the spongy inner layer of bone, which is lighter and less dense than cortical bone. The term “trabecula” refers to the microscopic structural unit of spongy bone, which is of a rod-like shape and collagenous composition.

Bone is a dynamic tissue that undergoes constant remodeling. The term “osteoblast” refers to a terminally-differentiated bone forming cell that deposits osteoid. The term “osteoid” refers to immature, unmineralized bone that is comprised primarily of type-I collagen. The term “pre-osteoblast” refers to a proliferating immature osteoblast that is not fully differentiated. The term “osteoprogenitor” refers to a pluripotent cell that gives rise to several stromal cell types, including osteoblasts. Osteoprogenitor cells, which are commonly referred to as “mesenchymal stem cells,” arise in the bone marrow and can be isolated in small numbers from circulating blood. The term “osteoclast” refers to a multinucleated, terminally-differentiated bone resorbing cell that is descended from a bone marrow monocyte. The term “pre-osteoclast” refers to a uninucleate and proliferating immature osteoclast. Osteoclasts are formed by the fusion of several pre-osteoclasts. Pre-osteoclasts can be identified by nuclear localization of the protein nuclear factor of activated T-cells (NFAT), the transcriptional activator of receptor activator of nuclear factor κB (RANK) intracellular signaling. The binding of RANK ligand (RANKL) to RANK is the key mediator of osteoclast differentiation and maturation. Mature osteoclasts can be identified by the expression of CD51/61 or tartrate resistant acid phosphatase (TRAP), and by their flat, multinucleated appearance.

Under normal homeostatic conditions, osteoblasts and osteoclasts work in unison to maintain bone integrity. Humoral factors cause osteoblasts to proliferate and functionally differentiate, resulting in bone deposition. In turn, osteoblast secreted factors, such as RANKL stimulate osteoclastogensis and bone resorption. Pathology results when bone deposition and bone resorption become uncoupled. For example, osteopetrosis is a bone disease characterized by overly dense, hard bone that is a result of unresorptive osteoclasts, while osteoporosis is a bone disorder characterized by brittle, porous bones, which can result from increased osteoclast activity.

Certain cancers, such as breast cancer and prostate cancer, have a propensity for metastasizing to bone where they disrupt bone homeostasis. Metastases of prostate cancer tend to be osteoblastic in nature while metastases of breast cancer are typically osteolytic. Metastatic breast cancers secrete several cytokines and growth factors that are known to affect osteoblast and osteoclast differentiation and function, including granulocyte macrophage colony stimulating factor (GMCSF), macrophage colony stimulating factor (MCSF), osteopontin (OPN), bone sialoprotein (BSP), parathyroid hormone related peptide (PTHrP), and interleukin-8 (IL-8).

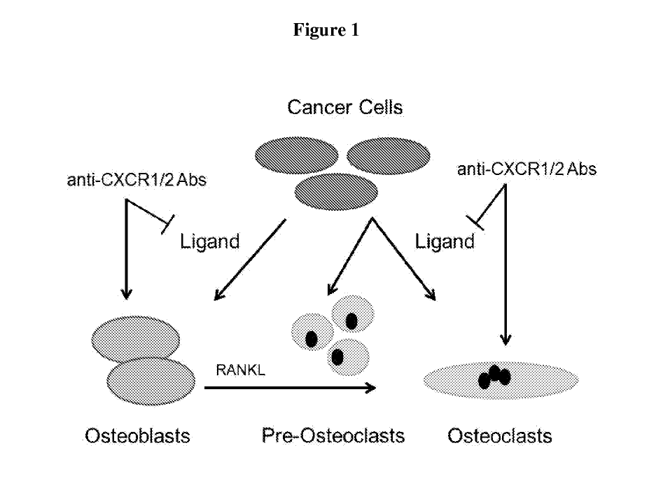

IL-8 is a secreted cytokine that is a local mediator of inflammation. Two of the most common receptors of IL-8 ligand are the G protein-coupled receptors CXCR1 and CXCR2. While CXCR2 is known to bind IL-8 as well as other ligands, IL-8 is the sole ligand to which CXCR1 is known to bind. Tumor cell secreted IL-8 is known to increase RANKL expression in osteoblasts as well as promote the proliferation and differentiation of pre-osteoclasts and the activity of mature osteoclasts. FIG. 1 provides a schematic illustration of the putative mechanism of IL-8-mediated bone resorption and inhibition with anti-CXCR1/2 antibodies. The present disclosure includes compositions and methods of inhibiting osteoclast differentiation and activity with anti-CXCR1/2 antibodies.

Several methods can be used to measure and characterize the structure, density, and quality of bone, including histology and histomorphometry, atomic force microscopy, confocal Raman microscopy, nanoindentation, three-point bending test, X-ray imaging, and micro computed tomography (μ-CT). In an exemplified embodiment, bones are measured and characterized by at least one of these methods.

The term “bone volume density” refers to the fraction of a given volume of bone (total volume or TV) that is comprised of calcified matter (bone volume or BV). Therefore, bone volume density is calculated as BV/TV and reported as a percentage. The term “specific bone surface” refers to the total bone surface (BS) per given volume of bone. Therefore, specific bone surface is calculated as BS/TV. Other common bone measurements include: bone area (B.Ar), trabecular number (Tb.N); trabecular spacing (Tb.Sp); osteoclast number (N.Oc); osteoclast surface area (Oc.S); osteoclasts per total bone surface (Oc.S/BS); osteoblast number (N.Ob), osteoblast surface area (Ob.S), osteoblast perimeter (Ob.Pm), and derivatives of any of said measurements. A larger Oc.S/BS is an indicator of increased bone resorption by osteoclasts.

### C. CXC Chemokine Receptors

The CXC chemokine receptor family comprises seven members (CXCR1-CXCR7) that share the common structural feature of spanning the cell membrane seven times (7-TM). CXC chemokine receptors mediate a variety of cellular functions, including cell homing and the upregulation and secretion of cytokines. Human CXCR1 and CXCR2 (Swiss Prot accession numbers P25024 and P25025, respectively) are closely related monomeric G protein-coupled receptors which, in their biologically active state, bind IL-8. The 350 amino acid sequence of CXCR1 and 360 amino acid sequence of CXCR2 are represented by SEQ ID NO: 1 and SEQ ID NO: 2, respectively.

| hCXCR1 | |
|---|---|
| (SEQ ID NO: 1) | |
| MSNITDPQMW DFDDLNFTGM PPADEDYSPC MLETETLNKY VVIIAYALVF LLSLLGNSLV | 60 |
| MLVILYSRVG RSVTDVYLLN LALADLLFAL TLPIWAASKV NGWIFGTFLC KVVSLLKEVN | 120 |
| FYSGILLLAC ISVDRYLAIV HATRTLTQKR HLVKFVCLGC WGLSMNLSLP FFLFRQAYHP | 180 |
| NNSSPVCYEV LGNDTAKWRM VLRILPHTFG FIVPLFVMLF CYGFTLRTLF KAHMGQKHRA | 240 |
| MRVIFAVVLI FLLCWLPYNL VLLADTLMRT QVIQESCERR NNIGRALDAT EILGFLHSCL | 300 |
| NPIIYAFIGQ NFRHGFLKIL AMHGLVSKEF LARHRVTSYT SSSVNVSSNL | 350 |
| hCXCR2 | |
| (SEQ ID NO: 2) | |
| MEDFNMESDS FEDFWKGEDL SNYSYSSTLP PFLLDAAPCE PESLEINKYF VVIIYALVFL | 60 |
| LSLLGNSLVM LVILYSRVGR SVTDVYLLNL ALADLLFALT LPIWAASKVN GWIFGTFLCK | 120 |
| VVSLLKEVNF YSGILLLACI SVDRYLAIVH ATRTLTQKRY LVKFICLSIW GLSLLLALPV | 180 |
| LLFRRTVYSS NVSPACYEDM GNNTANWRML LRILPQSFGF IVPLLIMLFC YGFTLRTLFK | 240 |
| AHMGQKHRAM RVIFAVVLIF LLCWLPYNLV LLADTLMRTQ VIQETCERRN HIDRALDATE | 300 |
| ILGILHSCLN PLIYAFIGQK FRHGLLKILA IHGLISKDSL PKDSRPSFVG SSSGHTSTTL | 360 |

### D. Molecules that Bind to CXCR1/2

The present invention includes methods that comprise administering to a subject one or more molecules that bind to CXCR1/2. The CXCR1/2 binders may be any binding molecule, such as an antibody, a fusion protein (e.g., an immunoadhesin), an siRNA, a nucleic acid, an aptamer, a protein, or a small molecule organic compound. For example, the invention includes one molecule that binds to both CXCR1 and CXCR2. Alternatively, the invention includes two molecules wherein one molecule binds to CXCR1 and a second molecule binds to CXCR2.

In certain embodiments, the invention includes one or more antibodies that bind to CXCR1/2 (anti-CXCR1/2 antibodies), or variants thereof, or antigen binding fragments thereof. For example, the invention includes one antibody, such as a bispecific antibody or a pan-specific antibody that binds to both CXCR1 and CXCR2. Alternatively, the invention includes two antibodies, wherein one antibody binds to CXCR1 and a second antibody binds to CXCR2. Anti-CXCR1/2 antibodies specifically bind to CXCR1/2 proteins, polypeptide fragments, or epitopes. The molecules that bind to CXCR1/2 may be from any species.

In certain exemplary embodiments, the antibodies that bind to CXCR1/2 are humanized antibodies, fully human antibodies, or variants thereof, or antigen-binding fragments thereof. Preferred anti-CXCR1/2 antibodies prevent binding of IL-8 with its receptors and/or inhibit CXCR1/2 biological activity (e.g., CXCR1/2 receptor-mediated osteoclastogenesis and osteoclast activity). In certain embodiments, the one or more antibodies, or antigen-binding fragments thereof, are CXCR1 and CXCR2 blocking antibodies (R&D Systems; Cat# MAB330 and MAB331, respectively).

In an exemplary embodiment of the invention, the antibodies that bind to CXCR1/2 are humanized or fully human antibodies. Examples of humanized and fully human antibody isotypes include IgA, IgD, IgE, IgG, and IgM. Preferably, the anti-CXCR1/2 antibodies are IgG antibodies. There are four forms of IgG. Preferably, the anti-CXCR1/2 antibodies are IgG2a antibodies. In one embodiment of the invention, the anti-CXCR1/2 antibodies are humanized IgG2a antibodies. In another embodiment of the invention, the anti-CXCR1/2 antibodies are fully human IgG2a antibodies.

As indicated above, certain embodiments of the invention also include variants or derivatives of anti-CXCR1/2 antibodies. Variants of anti-CXCR1/2 antibodies may have similar physicochemical properties based on their high similarity, and therefore are also included within the scope of the invention. Variants are defined as antibodies with an amino acid sequence that is at least 80%, at least 90%, at least 95%, or at least 97%, e.g., least 98% or 99% homologous to an anti-CXCR1/2 antibody described herein, and capable of competing for binding to a CXCR1/2 polypeptide, a CXCR1/2 polypeptide fragment, or a CXCR1/2 epitope. Preferably, the variants will ameliorate, neutralize, or otherwise inhibit binding of IL-8 with its CXCR1/2 receptors and CXCR1/2 biological activity (e.g., CXCR1/2 receptor-mediated osteoclast differentiation and activity). Determining competition for binding to the target can be done by routine methods known to the skilled person in the art. Preferably the variants are human antibodies, and preferably are IgG2a molecules. The term “variant” refers to an antibody that comprises an amino acid sequence that is altered by one or more amino acids compared to the amino acid sequences of the anti-CXCR1/2 antibody. The variant may have conservative sequence modifications, including amino acid substitutions, modifications, additions, and deletions.

Examples of modifications include, but are not limited to, glycosylation, acetylation, pegylation, phosphorylation, amidation, derivatization by known protecting/blocking groups, proteolytic cleavage, and linkage to a cellular ligand or other protein Amino acid modifications can be introduced by standard techniques known in the art, such as site-directed mutagenesis, molecular cloning, oligonucleotide-directed mutagenesis, and random PCR-mediated mutagenesis in the nucleic acid encoding the antibodies. Conservative amino acid substitutions include the ones in which the amino acid residue is replaced with an amino acid residue having similar structural or chemical properties. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, cysteine, tryptophan), nonpolar side chains (e.g., glycine, alanine, valine, leucine, isoleucine, proline, phenylalanine, methionine), beta-branched side chains (e.g., threonine, valine, isoleucine), and aromatic side chains (e.g., tyrosine, phenylalanine, tryptophan). It will be clear to the skilled artisan that classifications of amino acid residue families other than the one used above can also be employed. Furthermore, a variant may have non-conservative amino acid substitutions, e.g., replacement of an amino acid with an amino acid residue having different structural or chemical properties. Similar minor variations may also include amino acid deletions or insertions, or both. Guidance in determining which amino acid residues may be substituted, modified, inserted, or deleted without abolishing immunological activity may be found using computer programs well known in the art. Computer algorithms, such as, inter alia, Gap or Bestfit, which are known to a person skilled in the art, can be used to optimally align amino acid sequences to be compared and to define similar or identical amino acid residues. Variants may have the same or different, either higher or lower, binding affinities compared to an anti-CXCR1/2 antibody, but are still capable of specifically binding to CXCR1/2, and may have the same, higher or lower, biological activity as the anti-CXCR1/2 antibody.

Embodiments of the invention also include antigen binding fragments of the anti-CXCR1/2 antibodies. The terms “antigen binding domain,” “antigen binding region,” “antigen binding fragment,” and similar terms refer to that portion of an antibody that comprises the amino acid residues that interact with an antigen and confer on the binding agent its specificity and affinity for the antigen (e.g., the complementarity determining regions (CDR)). The antigen binding region can be derived from any animal species, such as rodents (e.g., rabbit, rat or hamster) and humans. Preferably, the antigen binding region will be of human origin. Non-limiting examples of antigen binding fragments include: Fab fragments, F(ab′)2 fragments, Fd fragments, Fv fragments, single chain Fv (scFv) molecules, dAb fragments, and minimal recognition units consisting of the amino acid residues that mimic the hypervariable region of the antibody.

### E. Therapeutic Administration

The compositions and methods described herein comprise administering a therapeutically effective amount of one or more molecules that bind to CXCR1/2 to a subject. As used herein, the phrase “therapeutically effective amount” means a dose of one or more molecules that binds to CXCR1/2 that results in a detectable improvement in one or more symptoms associated with pathologic osteolysis or which causes a biological effect (e.g., a decrease in the level of a particular biomarker) that is correlated with the underlying pathologic mechanism(s) giving rise to the condition or symptom(s) of osteolytic bone loss. For example, a dose of one or more molecules that bind to CXCR1/2 that increases bone mineral density, increases bone mass and/or bone strength, reduces bone fractures, and/or improves any diagnostic measurement of pathologic osteolysis is deemed a therapeutically effective amount.

In an embodiment, bone mineral density, bone mass, and/or bone strength are increased by about 5% to about 200% following treatment with one or more antibodies that bind to CXCR1/2. In certain embodiments, bone mineral density, bone mass, and/or bone strength are increased by about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 100%, about 100% to about 105%, about 105% to about 110%, about 110% to about 115%, about 115% to about 120%, about 120% to about 125%, about 125% to about 130%, about 130% to about 135%, about 135% to about 140%, about 140% to about 145%, about 145% to about 150%, about 150% to about 155%, about 155% to about 160%, about 160% to about 165%, about 165% to about 170%, about 170% to about 175%, about 175% to about 180%, about 180% to about 185%, about 185% to about 190%, about 190% to about 195%, or about 195% to about 200%, following treatment with one or more molecules that bind CXCR1/2.

In certain embodiments, a dose of molecules that reduces serum biomarkers of bone resorption, such as urinary hydroxyproline, urinary total pyridinoline (PYD), urinary free deoxypyridinoline (DPD), urinary collagen type-I cross-linked N-telopeptide (NTX), urinary or serum collagen type-I cross-linked C-telopeptide (CTX), bone sialoprotein (BSP), osteopontin (OPN), and tartrate-resistant acid phosphatase 5b (TRAP), is deemed a therapeutically effective amount. In an embodiment, serum biomarkers of bone resorption are reduced by about 5% to about 200% following treatment with one or more molecules that binds to CXCR1/2.

In an embodiment, serum biomarkers of bone resorption, such as urinary hydroxyproline, urinary total pyridinoline (PYD), urinary free deoxypyridinoline (DPD), urinary collagen type-I cross-linked N-telopeptide (NTX), urinary or serum collagen type-I cross-linked C-telopeptide (CTX), bone sialoprotein (BSP), osteopontin (OPN), and tartrate-resistant acid phosphatase 5b (TRAP), are decreased by about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 100%, about 100% to about 105%, about 105% to about 110%, about 110% to about 115%, about 115% to about 120%, about 120% to about 125%, about 125% to about 130%, about 130% to about 135%, about 135% to about 140%, about 140% to about 145%, about 145% to about 150%, about 150% to about 155%, about 155% to about 160%, about 160% to about 165%, about 165% to about 170%, about 170% to about 175%, about 175% to about 180%, about 180% to about 185%, about 185% to about 190%, about 190% to about 195%, or about 195% to about 200%, following treatment with one or more molecules that bind to CXCR1/2.

In certain embodiments, a dose of one or more molecules that increases serum biomarkers of bone deposition, such as total alkaline phosphatase, bone-specific alkaline phosphatase, osteocalcin, and type-I procollagen (C-terminal/N-terminal), is deemed a therapeutically effective amount. In an embodiment, serum biomarkers of bone deposition are increased by about 5% to about 200% following treatment with one or more molecules that bind to CXCR1/2.

In an embodiment, serum biomarkers of bone deposition, such as total alkaline phosphatase, bone-specific alkaline phosphatase, osteocalcin, and type-I procollagen (C-terminal/N-terminal), are increased by about 5% to about 10%, about 10% to about 15%, about 15% to about 20%, about 20% to about 25%, about 25% to about 30%, about 30% to about 35%, about 35% to about 40%, about 40% to about 45%, about 45% to about 50%, about 50% to about 55%, about 55% to about 60%, about 60% to about 65%, about 65% to about 70%, about 70% to about 75%, about 75% to about 80%, about 80% to about 85%, about 85% to about 90%, about 90% to about 95%, about 95% to about 100%, about 100% to about 105%, about 105% to about 110%, about 110% to about 115%, about 115% to about 120%, about 120% to about 125%, about 125% to about 130%, about 130% to about 135%, about 135% to about 140%, about 140% to about 145%, about 145% to about 150%, about 150% to about 155%, about 155% to about 160%, about 160% to about 165%, about 165% to about 170%, about 170% to about 175%, about 175% to about 180%, about 180% to about 185%, about 185% to about 190%, about 190% to about 195%, or about 195% to about 200%, following treatment with one or more molecules that bind to CXCR1/2.

Other embodiments include administering a therapeutically effective dose of one or more molecules for treating metastatic cancers. For example, a dose of one or more molecules that bind to CXCR1/2 that prevents the growth of bone metastases is deemed a therapeutically effective amount. In another example, a dose of one or more molecules that bind to CXCR1/2 that improves any symptom of a subject with bone metastases is deemed a therapeutically effective amount.

In accordance with the methods of the present invention, a therapeutically effective amount of one or more molecules that bind to CXCR1/2 that is administered to a subject will vary depending upon the age and the size (e.g., body weight or body surface area) of the subject, as well as the route of administration, and other factors well known to those of ordinary skill in the art.

In certain exemplary embodiments, the one or more anti-CXCR1/2 antibodies are administered to the subject as a subcutaneous dose. Other exemplary modes of administration include, but are not limited to, intradermal, intramuscular, intraperitoneal, intravenous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. The one or more CXCR1/2 antibodies can be administered parenterally or subcutaneously.

Various delivery systems are known and can be used to administer the pharmaceutical composition, e.g., encapsulation in liposomes, microparticles, microcapsules, receptor mediated endocytosis (see, e.g., Wu et al. (1987) J. Biol. Chem. 262:4429-4432). The therapeutic compositions will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Pharmaceutical compositions may be prepared into dosage forms in a unit dose suited to fit a dose of the active ingredients. Such dosage forms in a unit dose include, for example, tablets, pills, capsules, injections (ampoules), suppositories, etc.

Pharmaceutical compositions can also be administered to the subject using any acceptable device or mechanism. For example, the administration can be accomplished using a syringe and needle or with a reusable pen and/or autoinjector delivery device. The methods of the present invention include the use of numerous reusable pen and/or autoinjector delivery devices to administer a CXCR1/2 binder (or pharmaceutical formulation comprising the binder). Examples of such devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™ OPTIPEN STARLET™, and OPTICLIK™ (sanofi-aventis, Frankfurt, Germany), to name only a few. Examples of disposable pen and/or autoinjector delivery devices having applications in subcutaneous delivery of a pharmaceutical composition include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park, Ill.), to name only a few.

The use of a microinfusor to deliver a CXCR1/2 binder (or pharmaceutical formulation comprising the binder) to a subject is also contemplated herein. As used herein, the term “microinfusor” means a subcutaneous delivery device designed to slowly administer large volumes (e.g., up to about 2.5 mL or more) of a therapeutic formulation over a prolonged period of time (e.g., about 10, 15, 20, 25, 30 or more minutes). See, e.g., U.S. Pat. No. 6,629,949; U.S. Pat. No. 6,659,982; and Meehan et al., J. Controlled Release 46:107-116 (1996). Microinfusors are particularly useful for the delivery of large doses of therapeutic proteins contained within high concentration (e.g., about 100, 125, 150, 175, 200 or more mg/mL) and/or viscous solutions.

### F. Combination Therapies

Combination therapies may be employed for the purpose of reducing acquired resistance, reducing the dose of any one or more of the combined therapeutics in order to achieve efficacy with improved toxicities, to sensitize cells to one or more members of the combined therapy, or to achieve additive or greater than additive (e.g., synergistic) effects compared to the activities of the individual therapies. Computational methods of measuring additive and synergistic effects are routine and known to one of skill in the art. See, e.g., Fitzgerald, J. B. et al. (2006) Nat. Chem. Biol. 2(9):458-466, and Yan, H. et al, (2010) BMC Syst. Biol. 4:50.

In certain aspects, the invention includes methods for treating osteolytic bone diseases that comprise administering to a subject in need of such treatment one or more molecules that bind to CXCR1/2 in combination with at least one additional therapeutic agent. Examples of additional therapeutic agents that can be administered in combination with one or more anti-CXCR1/2 antibodies in the practice of the methods of the present invention include, but are not limited to, bisphosphonates, calcitonin, teriparatide, and any other compound known to treat, prevent, or ameliorate osteolytic bone diseases in a subject.

In the present methods, the additional therapeutic agent(s) can be administered concurrently or sequentially with the one or more molecules that bind to CXCR1/2. For example, for concurrent administration, a pharmaceutical formulation can be made that contains both molecules that bind to CXCR1/2 and at least one additional therapeutic agent.

In an embodiment, the one or more molecules that bind to CXCR1/2 are administered in combination with pharmaceutical bisphosphonates (e.g., Etidronate, Clodronate, Tiludronate, Pamidronate, Neridronate, Olpadronate, Alendronate, Ibandronate, Zoledronate, and Risedronate). In another embodiment, the one or more molecules that bind to CXCR1/2 are administered in combination with a drug that stimulates bone formation, such as parathyroid hormone analogs and calcitonin. In yet another embodiment, the one or more molecules that bind to CXCR1/2 are administered in combination with a selective estrogen receptor modulator (SERM). The amount of the additional therapeutic agent that is administered in combination with the one or more molecules that bind to CXCR1/2 in the practice of the methods of the present invention can be easily determined using routine methods known and readily available in the art.

### EXAMPLES

Osteolytic diseases are characterized by excessive bone resorption due to increased proliferation and differentiation of pre-osteoclasts and the activation of mature bone resorbing osteoclasts. IL-8 stimulates bone resorption by acting directly upon pre-osteoclasts and mature osteoclasts expressing CXCR1/2, and indirectly upon osteoblasts expressing CXCR1/2, thereby causing osteoblasts to upregulate expression of RANKL.

### Example 1

### Inhibition of IL-8 Blocks Osteoclast Differentiation

Treatment of osteolytic disease requires that a therapeutic be efficacious at blocking osteoclast formation regardless of the differentiation stage when exposed to the therapeutic. To investigate the effectiveness of simultaneous inhibition of both CXCR1 and CXCR2 throughout the osteoclast differentiation cascade, addition of blocking antibodies to both receptors was delayed by a range of intervals after initiation of differentiation.

### Example 1.1

Osteoclast precursor cells (Lonza; Cat#2T-110) were seeded in multi-well tissue culture plates and cultured in osteoclast precursor medium (Lonza; Cat# PT-8001) supplemented according to the manufacturer's instructions. To induce osteoclast differentiation, the culture medium was supplemented with MCSF (33 ng/mL) and RANKL (33 ng/mL). CXCR1 (R&D Systems; Cat# MAB330), CXCR2 (R&D Systems; Cat# MAB331), and IL-8 blocking antibodies were added daily to each well by adding 5 μL of 40× concentrated antibodies to achieve a final antibody concentration of 30 μg/mL, 30 μg/mL, and 50 μg/mL, respectively, on experimental days 0-6, 1-6, 2-6, 3-6, 4-6, or 5-6. The cultures were fixed with 3.7% paraformaldehyde on experimental day 6 and stained for markers of differentiating and mature osteoclasts using an antibody against NFATc1, a transcription factor that regulates osteoclastogenesis (Santa Cruz; Cat# sc-13033) and anti-CD51/CD61 (BD Biosciences; Cat#550037) antibodies, respectively. Images were acquired on an Applied Precision Arrayworx Multiformat Reader and segmented and quantified using ImageRail public domain software with an osteoclast segmentation algorithm.

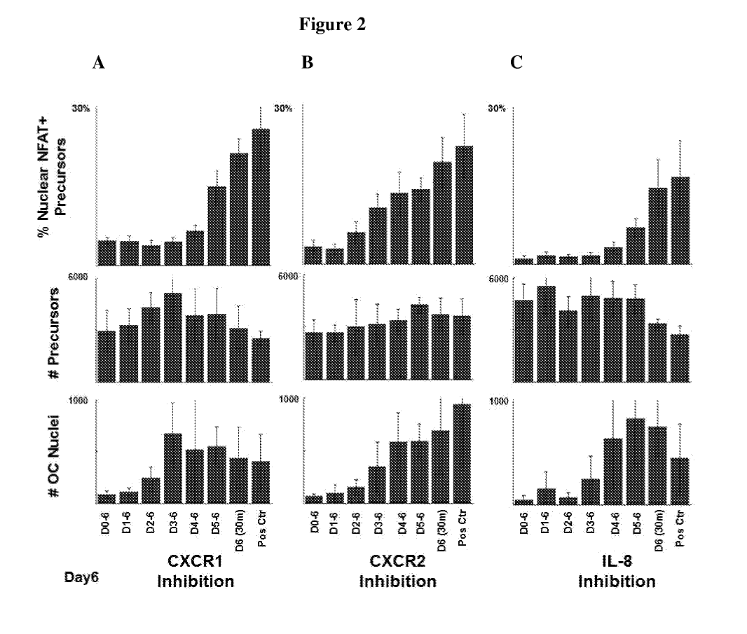

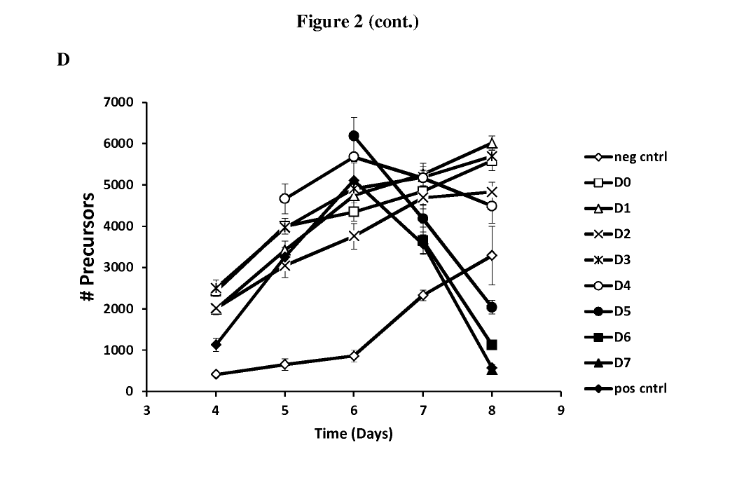

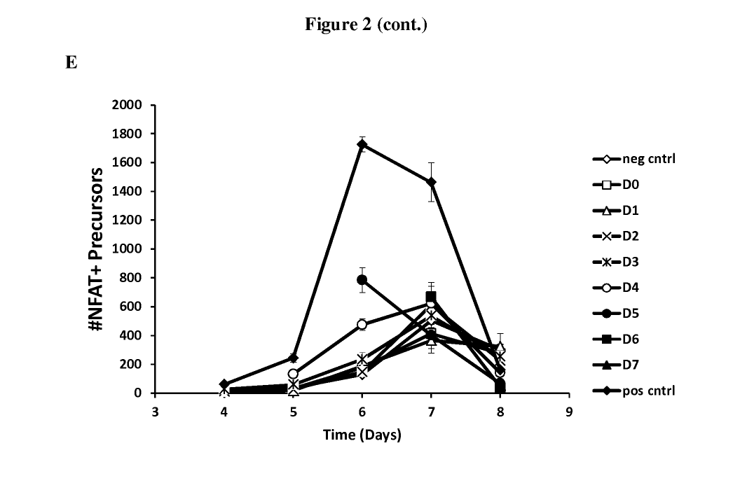

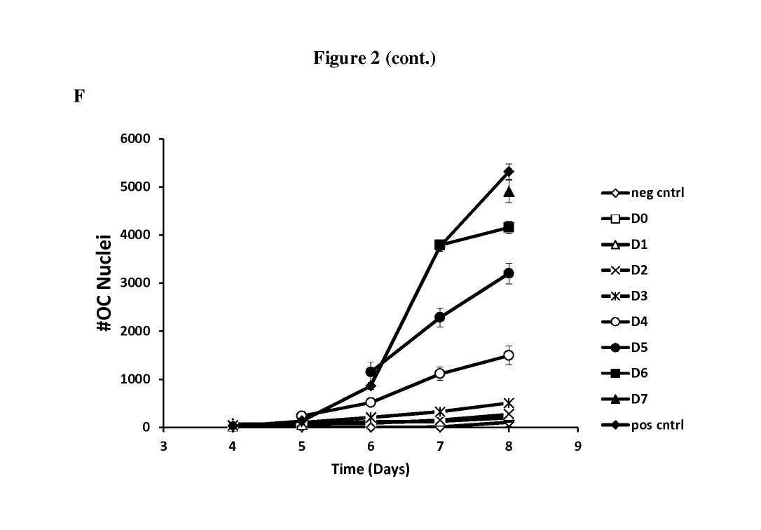

As can be seen in FIG. 2A-C, treatment with all blocking antibodies resulted in fewer pre-osteoclasts and mature multinucleated osteoclasts with the greatest effect observed in treatment groups that were treated daily beginning on days 0, 1, or 2. Inhibition of IL-8 resulted in fewer NFAT positive pre-osteoclasts than inhibition of either CXCR1 or CXCR2, suggesting that blocking IL-8 ligand is more effective at inhibiting pre-osteoclasts than blocking either of the IL-8 receptors alone. These data suggest that there is a time-delay in IL-8 inhibition, and that inhibitors must be added on days 0 through 2 of osteoclast differentiation to prevent the fusion and maturation of osteoclasts.

### Example 1.2

All conditions contained 33 ng/mL of MCSF and 33 ng/mL of RANKL with the exception of the negative control (neg cntl) which did not contain RANKL. The positive control (pos cntl) contained RANKL but did not contain antibodies. Treatments consisted of a combination of CXCR1 antibody (45 μg/mL, final concentration) and CXCR2 antibody (45 m/mL, final concentration) added daily as 5 μL of 40× concentrated antibody solution per the schedule in Table 1. Medium was not changed during the experiment. Cultures were fixed on days 4, 5, 6, 7 and 8.

TABLE 1
CXCR1 and CXCR2 antibodies added at 40x final concentration on days indicated
for cultures fixed on Day 8. For cultures fixed on earlier days, the same
schedule was used, but no antibodies were added on the day of fixation.
No antibodies added for negative or positive controls.

| | | Antibody Addition on: | | | | | | | |
|---|---|---|---|---|---|---|---|---|---|
| | | Day 0 | Day 1 | Day 2 | Day 3 | Day 4 | Day 5 | Day 6 | Day 7 |
| Treatment: | D 0 | X | X | X | X | X | X | X | X |
| | D 1 | | X | X | X | X | X | X | X |
| | D 2 | | | X | X | X | X | X | X |
| | D 3 | | | | X | X | X | X | X |
| | D 4 | | | | | X | X | X | X |
| | D 5 | | | | | | X | X | X |
| | D 6 | | | | | | | X | X |
| | D 7 | | | | | | | | X |

neg cntl
pos cntl

Regardless of the day of CXCR1+CXCR2 antibody addition, osteoclast fusion was reduced as shown by a reduction in the number of NFAT positive precursors 24 hours after antibody addition (FIG. 2E) and a reduction in the number of osteoclast nuclei 48 hours after antibody addition (FIG. 2F). All data represented as mean±SEM. The combination of CXCR1 and CXCR2 antibody addition also increases the number of undifferentiated osteoclast precursors compared to the positive control with a larger increase when antibodies are added earlier in culture (FIG. 2D).

### Example 2

### Equimolar Mixture of Anti-CXCR1 and Anti-CXCR2 Antibodies Inhibit Osteoclast Differentiation

Osteoclast precursor cells (Lonza; Cat#2T-110) were seeded in 96-well tissue culture plates and cultured in osteoclast precursor medium (Lonza; Cat# PT-8001) supplemented according to the manufacturer's instructions. To induce osteoclast differentiation, the culture medium was supplemented with MCSF (33 ng/mL) and RANKL (33 ng/mL) and 200 μL of medium was added per well. CXCR1 and CXCR2 blocking antibodies (R&D Systems; Cat# MAB330 and MAB331, respectively) were reconstituted per the product specifications and diluted to 40 times the final concentration (2000, 500, 125, and 31.25 μg/mL) either alone or in combination. 5 uL of 40× concentrated antibodies were added to each well on experimental days 0, 1, 2, 3, 4, 5, and 6 for a final antibody concentration of 50, 12.5, 3.125, and 0.78 μg/mL, respectively. Each condition was repeated in 6 independent wells. The cultures were fixed with 3.7% paraformaldehyde on experimental day 7 and stained for markers of differentiating and mature osteoclasts using anti-NFATc1 (Santa Cruz; Cat# sc-13033) and anti-CD51/CD61 (BD Biosciences; Cat#550037) antibodies, respectively. Images were acquired (9 fields/well) on an Applied Precision Arrayworx Multiformat Reader and segmented and quantified using ImageRail public domain software with an osteoclast segmentation algorithm. Data were plotted as mean per well (n=6 wells+/−SEM).

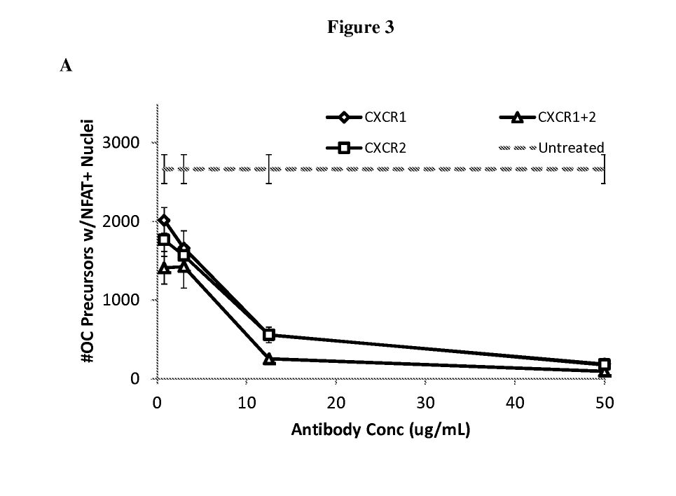

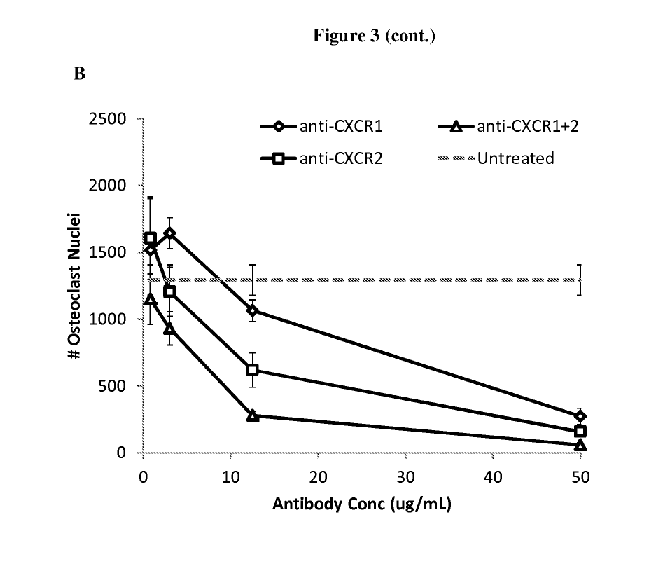

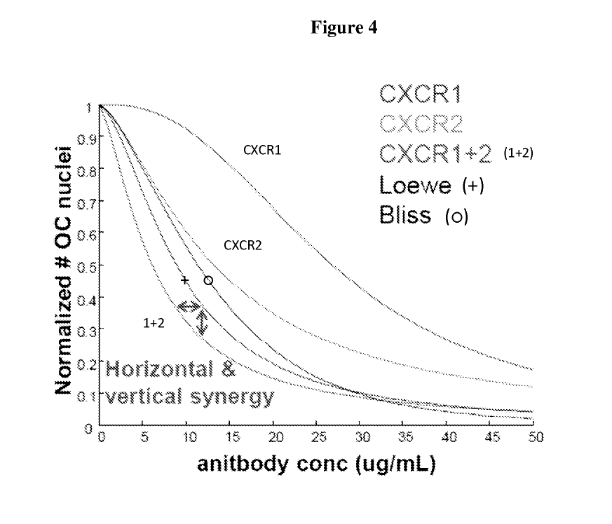

As shown in FIG. 3, combined treatment with anti-CXCR1 and anti-CXCR2 antibodies resulted in fewer differentiating osteoclasts and mature osteoclasts compared to treatment with either antibody alone. This effect was observed at all concentrations tested with the greatest effect observed in the 12.5-0.78 μg/mL concentration range. These results suggest that CXCR1 and CXCR2 activity may act synergistically in the osteoclast differentiation pathway. To determine whether anti-CXCR1 and anti-CXCR2 antibodies act synergistically, Loewe additivity and Bliss independence were calculated from antibody dose response curves, according to the methods described by Fitzgerald et al (See, e.g., Fitzgerald, J. B. et al. (2006) Nat. Chem. Biol. 2(9):458-466). As demonstrated in FIG. 4, the Loewe's additivity curve appears to the left of the Bliss independence curve and to the right of the anti-CXCR1/2 dose response curve, indicating that the combination of anti-CXCR1 and anti-CXR2 antibodies act synergistically through a common pathway. The Loewe's additivity curve (+) appears to the left of the Bliss independence curve (o) and to the right of the anti-CXCR1/2 dose response curve “1+2”. The CXCR1 and CXCR2 curves are both to the right of the other curves. These results indicate that combination therapy with anti-CXCR1 and anti-CXR2 antibodies provides a synergistic improvement and that the antibodies act through a common pathway. As described in Fitzgerald, J. B. et al. (2006) Nat. Chem. Biol. 2(9):458-466 (which also details the analysis of Loewe's additivity and Bliss independence), such synergy can be viewed in two ways: vertical synergy, where the combination therapy involves the same doses as monotherapy (but with greater efficacy), and horizontal synergy, where the concentrations of the two inhibitors can be adjusted downward so as to achieve a constant level of combined efficacy. The horizontal and vertical arrows in this figure indicate that both horizontal and vertical synergy are produced by this combination.

Together, these examples confirm that the CXCR1 and CXCR2 receptors may be targeted to prevent osteoclastogenesis and osteolysis.

### EQUIVALENTS

Those skilled in the art will recognize, or be able to ascertain using no more than routine experimentation, many equivalents of the specific embodiments of the invention(s) described herein. Such equivalents are intended to be encompassed by the following claims Any combination of one or more of the embodiments disclosed in any independent claim and any of the dependent claims is also contemplated to be within the scope of the invention.

### INCORPORATION BY REFERENCE

Each and every patent, pending patent application, and publication referred to herein is hereby incorporated herein by reference in its entirety.

What is claimed is:

1. A composition comprising one or more molecules that specifically bind to CXC chemokine receptor 1 (CXCR1) and CXC chemokine receptor 2 (CXCR2).

2. The composition of claim 1, wherein at least one of the molecules is an antibody or an antigen binding fragment thereof.

3. The composition of claim 2, wherein the antibody is a bispecific antibody or a pan-specific antibody or an antigen binding fragment thereof.

5. The composition of claim 2, wherein the composition comprises two antibodies or antigen binding fragments thereof.

6. The composition of claim 1, wherein the one or more molecules act synergistically to inhibit a common intracellular signaling pathway.

7. The composition of claim 1, wherein the composition prevents interleukin-8 or another CXCR ligand from binding to CXCR1 and/or CXCR2.

8. The composition of claim 1, wherein the composition inhibits the activity of CXCR1 and/or CXCR2.

9. The composition of claim 1, wherein the composition inhibits osteoclast differentiation and/or activity.

10. The composition of claim 1, wherein the composition promotes osteoblast activity.

11. The composition of claim 1, wherein the composition prevents bone resorption and/or promotes bone deposition.

12. The composition of claim 1, wherein the composition inhibits the growth of bone metastases in a subject.

13. The composition of claim 1, wherein the composition further comprises an additional therapeutic agent.

14. The composition of claim 14, wherein the additional therapeutic agent is selected from the group consisting of a bisphosphonate, calcitonin, teriparatide, a parathyroid hormone analog, calcitonin, and a selective estrogen receptor modulator.

15. A method for treating osteolysis in a subject in need thereof comprising administering to the subject a therapeutically effective amount of the composition of any one of the previous claims.

16. The method of claim 15, wherein, following administration, the composition improves a bone parameter selected from the group consisting of bone volume density (BV/TV), total bone surface (BS), bone surface density (BS/BV), trabecular number (Tb.N), trabecular thickness (Tb.Th), trabecular spacing (Tb.Sp), and total volume (Dens TV).

17. The method of any one of claims 15-16, wherein, following administration, the composition reduces a serum biomarker of bone resorption selected from the group consisting of urinary hydroxyproline, urinary total pyridinoline (PYD), urinary free deoxypyridinoline (DPD), urinary collagen type-I cross-linked N-telopeptide (NTX), urinary or serum collagen type-I cross-linked C-telopeptide (CTX), bone sialoprotein (BSP), osteopontin (OPN), and tartrate-resistant acid phosphatase 5b (TRAP).

18. The method of any one of claims 15-17, wherein, following administration, the composition increases a serum biomarker of bone deposition selected from the group consisting of total alkaline phosphatase, bone-specific alkaline phosphatase, osteocalcin, and type-I procollagen (C-terminal/N-terminal).

19. The method of any one of claims 15-18, wherein, following administration, the composition inhibits bone resorption.

20. The method of any one of claims 15-19, wherein, following administration, the composition promotes bone deposition.

21. The method of any one of claims 15-20, wherein, following administration, the composition inhibits the growth of bone metastases in a subject.

22. The method of any one of claims 15-21, wherein, following administration, the composition improves a symptom of a subject with bone metastases.

23. The method of any one of claims 15-22, wherein the administering of the composition is by a parenteral or an oral route.

24. The method of claim 23 wherein the parenteral route is a subcutaneous, intradermal, intramuscular, intraperitoneal, intravenous, intranasal, intrathecal, inhalation, or intrarticular route.
